(19) United States
(12) Patent Application Publication
(10) Pub. No.: US 2026/0245742 A1
(43) Pub. Date: Aug. 20, 2026

(54) TRANSPARENT PATIENT SYMPTOM-MITIGATION CLUSTERING FROM TEXTUAL DATA

(71) Applicant: NEC Laboratories Europe GmbH, Heidelberg (DE)

(72) Inventors: Anja MOESCH, Heidelberg (DE); Carolin LAWRENCE, Heidelberg (DE)

(21) Appl. No.: 19/161,373

(22) PCT Filed: Nov. 21, 2023
(86) PCT No.: PCT/IB2023/061729

§ 371 Date: Sep. 2, 2025

Related U.S. Application Data
(60) Provisional application No. 63/538,515, filed on Sep. 15, 2023.

(51) Int. Cl. G16H 50/70; G16H 10/60; G16H 20/00; G16H 70/20

(52) U.S. Cl. CPC G16H 50/70; G16H 10/60; G16H 20/00; G16H 70/20

(57) ABSTRACT

A computer-implemented, machine learning (ML) method for determining optimized patient symptom-mitigation predictions includes receiving, from a user, profile information and user symptoms. The profile information and the user symptoms are matched to one of a plurality of profile clusters that are within one or more symptom-mitigation clusters created using textual data, the profile clusters being grouped within the one or more symptom-mitigation clusters based on profile information of authors of the textual data. A symptom-mitigation strategy is predicted based on the matching profile cluster. The method has applications including, but not limited to, use cases in medical artificial intelligence (AI)/healthcare for optimization of predictions or to support decision-making.

### CROSS-REFERENCE TO PRIOR APPLICATIONS

This application is a U.S. National Phase application under 35 U.S.C. § 371 of International Application No. PCT/IB2023/061729, filed on Nov. 21, 2023, and claims benefit to U.S. Provisional Application Ser. No. 63/538,515 filed on Sep. 15, 2023, the entire contents of which is hereby incorporated by reference herein. The International Application was published in English on Mar. 20, 2025 as WO 2025/056967 A1 under PCT Article 21(2).

### FIELD

The present invention relates to Artificial Intelligence (AI) and machine learning (ML), and in particular to a method, system, data structure, computer program product and computer-readable medium for determining patient symptom-mitigation predictions using clustering and crowd sourced data.

### SUMMARY

In an embodiment, the present invention provides a computer-implemented, machine learning (ML) method for determining optimized patient symptom-mitigation predictions. Profile information and user symptoms are received from a user. The profile information and the user symptoms are matched to one of a plurality of profile clusters that are within one or more symptom-mitigation clusters created using textual data, the profile clusters being grouped within the one or more symptom-mitigation clusters based on profile information of authors of the textual data. A symptom-mitigation strategy is predicted based on the matching profile cluster. The method has applications including, but not limited to, use cases in medical artificial intelligence (AI)/healthcare for optimization of predictions or to support decision-making.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 schematically illustrates an AI method and system for predicting diseases and/or mitigation strategies for symptoms according to an embodiment of the present invention;

FIG. 2 schematically illustrates an overview of the output structure from the profile-symptom-mitigation cluster creator according to an embodiment of the present invention;

FIG. 3 illustrates compiled information from the output creator according to an embodiment of the present invention;

FIG. 4 illustrates an example of a method and system for processing social media posts and user profiles to create symptom-mitigation clusters according to an embodiment of the present invention;

FIG. 5 illustrates an example output that could be presented to a user via a chatbot in a smartphone app according to an embodiment of the present invention; and

FIG. 6 is a block diagram of an exemplary processing system, which can be configured to perform any and all operations disclosed herein.

### DETAILED DESCRIPTION

A wealth of health advice is hidden in not curated and not peer reviewed social media posts written by a crowd of (usually) lay people. Embodiments of the present invention provide an AI method and system that leverages this information and maps social media recommendations to a user's symptoms in a reliable, trustworthy, explainable and transparent manner. This enables users to find out how their (e.g., minor) symptoms can be treated before or without the need of consulting a doctor or taking prescription medication. The AI method and system according to embodiments of the present invention provides for enhanced computer functionality by incorporating a special clustering technique that enables improved accuracy of the AI predictions, for example, enabling to identify a most promising mitigation candidate. Moreover, the AI method and system according to embodiments of the present invention has further enhanced computer functionality to provide the AI predictions that are personalized and more accurate and relevant for individual users.

For many health related issues and symptoms, it is not easy to identify the cause, disease, treatment recommendation or even to give general advice to reduce the symptoms. Especially for rare or unclear cases, there is not enough evidence in reliable publications on what treatment might help. Similarly, in some areas of the world, people might not have access to a doctor. There are also numerous cases of minor symptoms that can be easily treated with remedies available in almost every household, or specific activities and mitigation strategies, for which consulting a doctor or taking prescribed medication is unnecessary and costs significant amounts of time and resources. Additionally, “official” medical advice can be very generalized and often does not take individual parameters into account, like additional diagnoses, age and gender. However, at the same time, there is a wealth of information and advice from the crowd of lay people in the form of social media, such as forum posts, describing detailed user experiences and mitigation strategies for symptoms. Embodiments of the present invention are able to leverage this wealth of information to give an accurate, reliable, trustworthy, explainable and transparent recommendation for a user who described a particular profile and symptoms in a fast, automated and computational resource efficient manner.

Embodiments of the present invention provide an AI method and system that can help a user with a certain set of symptoms to find advice in the wealth of social media posts on how these symptoms can be mitigated. The AI method and system includes the enhanced computer functionality to do this in a transparent manner so that it is easy for the user to understand the reasoning process of the system and make an informed decision whether or not to try out a certain piece of advice.

In a first aspect, the present invention provides a computer-implemented, machine learning method for determining optimized patient symptom-mitigation predictions. Profile information and user symptoms are received from a user. The profile information and the user symptoms are matched to one of a plurality of profile clusters that are within one or more symptom-mitigation clusters created using textual data, the profile clusters being grouped within the one or more symptom-mitigation clusters based on profile information of authors of the textual data. A symptom-mitigation strategy is predicted based on the matching profile cluster.

In a second aspect, the present invention provides the method according to the first aspect, further comprising obtaining, from a data source, social media posts, and building a profile database including the profile information about the authors of the social media posts, wherein the data source includes the social media posts, the textual data, or crowd sourced data.

In a third aspect, the present invention provides the method according to the first aspect or the second aspect, further comprising building a symptom-mitigation database by extracting symptoms and mitigation strategies from the data source, wherein the one or more symptom-mitigation clusters are generated using a clustering algorithm, the profile information from the profile database, and the symptom-mitigation database.

In a fourth aspect, the present invention provides the method according to any of the first to third aspects, wherein the social media posts are selected from a social media database by classifying the social media posts by patients from patient groups based on content of the social media posts.

In a fifth aspect, the present invention provides the method according to any of the first to fourth aspects, further comprising computing, for each text entry of the matching profile cluster, similarity to text of the user symptoms, computing for each profile in the matching profile cluster, similarity to the profile information from the user, determining a combined ranking by combining the similarity to the text of the user symptoms and the similarity to the profile information from the user, and identifying a most common symptom-mitigation strategy based on the combined ranking.

In a sixth aspect, the present invention provides the method according to any of the first to fifth aspects, further comprising outputting to the user the most common symptom-mitigation strategy along with the textual data that links to the most common symptom-mitigation strategy and profile matches.

In a seventh aspect, the present invention provides the method according to any of the first to sixth aspects, wherein the combined ranking is determined by averaging a distance determined for the similarity to the text of the user symptoms and a distance determined for the similarity to the profile information from the user, and wherein different mitigation strategies are checked to determine if they are similar in order to identify the most common symptom-mitigation strategy.

In an eighth aspect, the present invention provides the method according to any of the first to seventh aspects, further comprising filtering the textual data based on whether the textual data contains information about how to mitigate a symptom.

In a ninth aspect, the present invention provides the method according to any of the first to eighth aspects, further comprising filtering out, modifying, or marking unsafe information in the textual data.

In a tenth aspect, the present invention provides the method according to any of the first to ninth aspects, wherein the clusters of profiles are generated based on prototypes.

In an eleventh aspect, the present invention provides the method according to any of the first to tenth aspects, wherein the one or more symptom-mitigation clusters are created using disease or symptom labels, wherein textual descriptions of symptoms are encoded as vectors using a large language model (LLM), and wherein the encoded vectors are used to train a clustering algorithm.

In a twelfth aspect, the present invention provides the method according to any of the first to eleventh aspects, further comprising concatenating the profile information for each of the authors of the textual data into a feature vector, which in each case is assigned to a mitigation strategy extracted from the textual data, and training a clustering model for each of the one or more symptom-mitigation clusters to generate the plurality of profile clusters in the one or more symptom-mitigation clusters.

In a thirteenth aspect, the present invention provides the method according to any of the first to twelfth aspects, further comprising generating a new feature vector based on a determination that a respective feature vector of the profile information of one of the authors has a distance to a centroid of one of the profile clusters that is greater than a threshold.

In a fourteenth aspect, the present invention provides a computer system for determining patient symptom-mitigation predictions comprising one or more processors, which, alone or in combination, are configured to perform a machine learning method for determining patient symptom-mitigation predictions according to any of the first to thirteenth aspects.

In a fifteenth aspect, the present invention provides a tangible, non-transitory computer-readable medium for determining patient symptom-mitigation predictions which, upon being executed by one or more hardware processors, provide for execution of a machine learning method according to any of the first to thirteenth aspects.

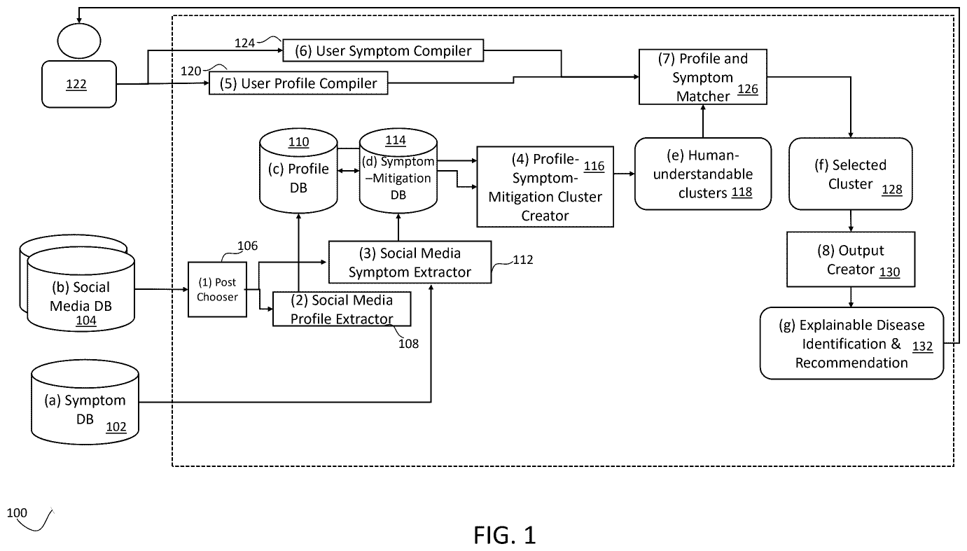

FIG. 1 schematically illustrates an AI method and system 100 for predicting diseases and/or mitigation strategies for symptoms according to an embodiment of the present invention. In FIG. 1 and the following text, a heading with a letter, e.g. (a), denotes a description of data (e.g., in a database (DB)), whereas a number, e.g. (1), denotes a component of the system). As used herein, diseases can refer to any health-related issue a person can experience with symptoms.

(a) Symptom DB 102: The symptom DB stores a list of possible symptoms based on the Unified Medical Language System (UMLS). This can also include information extraction from scientific publications or other medical resources.

(b) Social Media DB 104: The social media DB includes one or more databases (data sources) that store social media posts where people are likely to discuss health issues, symptoms and possible ways to mitigate symptoms. This data could, for example, be taken from forum posts or content provided by users on various social media platforms.

(1) Post Chooser 106: The post chooser 106 component takes a particular social media post (e.g., textual data) from the social media DB 104 and classifies whether or not it contains information about a possible way to mitigate a particular symptom. Example posts could be “Medication B makes me nauseous unless I eat beforehand” or “Eating oily foods helps with my constipation.” The post chooser 106 can perform the classification, for example, by utilizing a system that classifies medical posts by patients from patient groups according to their information content, thereby distinguishing reports of side effects from encouraging posts (see Anne Dirkson, Suzan Verberne, and Wessel Kraaij, “Narrative detection in online patient communities,” CEUR Workshop Proceedings, Leiden Institute of Advanced Computer Science, Leiden University (2019), which is hereby incorporated by reference herein). In embodiments the post chooser 106 may be implemented by a post chooser module or component. This step may also filter posts deemed dangerous. For this, each post is checked if any information is unsafe (e.g., if it contains toxic substances, harmful treatments etc.). If a post is determined to include unsafe content, the filter either directly removes it, modifies it to make it safe or marks it as potentially unsafe. A LLM could be used to determine unsafe content (e.g., detect harmful substances) in the text. The LLM can be trained to classify text of each post as either safe or unsafe. The data source may include textual data, for example from social media posts, or crowd sourced information.

(2) Social Media Profile Extractor 108: Once a particular post from the social media DB 104 has been chosen by the post chooser 106 component to be part of the system, the social media profile extractor 108 component looks up the author of the post and creates a profile for this person. The concrete instantiation of this extractor 108 depends on the social media DB 104 in the background and what information is already available. If possible, this is a direct lookup to retrieve profile information such as age and gender from the social media DB 104. If a direct lookup is not possible, a Large Language Model (LLM) is used to automatically assign the most likely profile information (age, gender, nationality, etc.) based on all the text written by this person. In embodiments, the social media profile extractor 108 can be implemented by a module or component. In an embodiment, a different LLM may be trained to automatically assign the most likely profile information based on all the text written by a person.

(c) Profile DB 110: The profile DB 110 contains a profile entry for each individual person and stores a series of pre-defined keys for each person. The pre-defined keys could, for example, be typical attributes such as age and gender, but can also include other attributes such as existing conditions or previous diagnoses. In an embodiment, another LLM can be trained to determine the pre-defined keys based on written text included in a post(s). Profiles generated by the social media profile extractor 108 component are stored in the profile DB 110, which can also include pre-existing patient profile information when available. Examples of pre-existing patient profile information can include information included in an electronic health record (EHR) for a given person/patient. The pre-existing patient profile information can include pre-existing conditions.

(3) Social Media Symptom Extractor 112: Once a particular post from the social media DB 104 has been chosen by the post chooser 106 component to be part of the system, the social media symptom extractor 112 component extracts all symptoms and corresponding mitigation strategy that can be found described in the post. The social media symptom extractor 112 component may use a model, such as an LLM, that is trained to identify symptoms and corresponding mitigation strategies from a post. For example, the model may be trained with sample labels and once trained, the model can be used to predict for each word in a post whether or not it is a symptom or mitigation strategy. If information is available to do so, the social media symptom extractor 112 component also rates on a scale (e.g., a 5 point Likert scale), how well the mitigation strategy worked. The same model described above or a new model implemented by the social media symptom extractor 112 component may be trained to classify words or phrases in a post to correspond to a rating scale such as the 5 point Likert scale. For example, a phrase included in a post such as “The method worked great!” may correspond to a 5 on the 5 point Likert scale. One example instantiation of this could be: use a Natural Language Processing (NLP) system, such as an existing LLM, and iterate over the words in a post and classify whether it describes a symptom. If it does, the social media symptom extractor 112 component extracts for this symptom the corresponding mitigation from the post, possibly with the scale, and saves this information in the symptom-mitigation database 114. If available, it can also extract the description of how well the mitigation strategy worked. Thus, posts can be classified on whether they contain relevant information, in particular a description of symptom mitigation (see Anne Dirkson, Suzan Verberne, Wessel Kraaij, Gerard van Oortmerssen, and Hans Gelderblom, “Automated gathering of real-world data from online patient forums can complement pharmacovigilance for rare cancers,” Scientific Reports, 12(1):10317 (2022), which is hereby incorporated by reference herein). For symptom identification, user language and terminology and symptom descriptions typically found in social media posts are mapped to a disease database using, for example, UMLS (see Sanja Scepanovic, Luca Maria Aiello, Ke Zhou, Sagar Joglekar, and Daniele Quercia, “The Healthy States of America: Creating a Health Taxonomy with Social Media,” Vol. 15 (2021): Fifteenth International AAAI Conference on Web and Social Media (2021); and Kerstin Denecke: “Extracting Medical Concepts from Medical Social Media with Clinical NLP Tools: A Qualitative Study”, University of Leipzig (2014), each of which is hereby incorporated by reference herein).

(d) Symptom-Mitigation Database 114: For each symptom extracted from a post, the symptom-mitigation database records the following information:
a) Textual description of the symptom from the post.
b) The post itself
c) The identified symptom as recorded in the symptom DB 102.
d) The mitigation strategy in textual form.
e) If available, how well the mitigation strategy worked.
f) The profile ID of the post author in the profile DB 110.

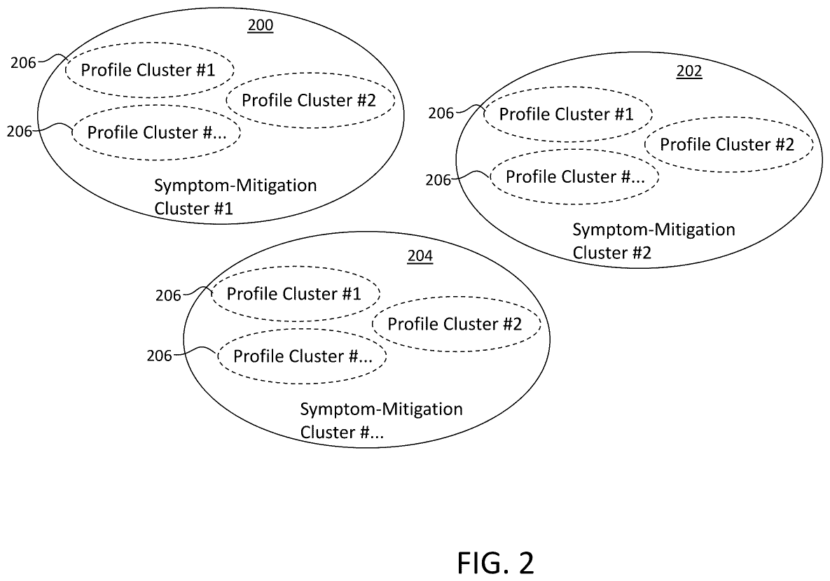

(4) Profile-Symptom-Mitigation Cluster Creator 116: The profile-symptom-mitigation cluster creator 116 component uses the entries of the symptom-mitigation database 114. Either each entry or a small seed set are annotated with labels that show which entries belong together. This could, for example, be an associated disease or symptom label, as could be directly extracted, for example, from Reddit sub-forums about certain diseases or from social media hashtags. Alternatively, a set of labels is provided and, for example, a LLM automatically determines in a zero-shot (e.g., using the LLM without prior labeled data) or few-shot (e.g., few labeled examples in prompts to adapt the LLM to new tasks) manner which entry belongs to which label. First, for each entry, the textual description of the symptom from the post is taken from the symptom-mitigation database 114 and encoded, for example with a LLM. The different textual descriptions could be combined in different ways (e.g., by concatenation). Based on the encoded vectors, a clustering algorithm is trained (e.g., based on prototype-based learning). The clustering algorithm could be any suitable clustering algorithm such as a K-nearest neighbors (KNN) algorithm. A distance measure is chosen which can be used to measure how far an input vector is away from each cluster centroid that has been learned. For example, the distance may be in a Euclidean feature space in which the clusters are embedded. This step creates an initial set of clusters with one cluster per label. This first set of clusters is also referred to herein as level 1. Next, the clusters are further divided by considering the different profiles within a cluster. This second set of clusters is also referred to herein as level 2. For this, all the information of each profile from the profile DB 110 is used and this information is concatenated into a feature vector. Each entry of the symptom-mitigation database 114 is assigned its corresponding profile vector build from the profile DB 110. For this level 2, a series of clustering models are trained, one for each textual cluster. The series of clustering models may be trained using the same clustering algorithm described above. A distance measure is chosen which can be used to measure how far an input vector is away from each cluster centroid that has been learned. This step splits each cluster from level 1 into further clusters, where each new centroid represents a typical patient in this cluster. In some embodiments, a new cluster can also be created. In this case, for any input vector that is still too far away from a cluster center (e.g. which has a distance larger than a specific threshold value that can be learned or preset), it is determined that a new cluster needs to be created for this vector. Given all such vectors, they are grouped together and a new cluster center is learned if they are close enough to each other. FIG. 2 illustrates exemplary clusters that can be output by the profile-symptom-mitigation cluster creator 116 component. In embodiments the profile-symptom-mitigation cluster creator 116 can be implemented by a component or module.

FIG. 2 depicts several symptom-mitigation clusters 200, 202, 204 corresponding to symptom-mitigation cluster #1, #2, and # . . ., respectively. Each symptom-mitigation cluster 200, 202, and 204 includes several profile clusters 206. Symptom-mitigation cluster #1 (200) may correspond to “migraine-icepack,” whereas symptom-mitigation cluster #2 (202) may correspond to “migraine-chamomile tea,” and symptom-mitigation cluster #3 (204) may correspond to “nausea-chamomile tea.” Each profile cluster 206 may represent groups of users with a common trait, for example one profile cluster 206 may include young women while another profile cluster 206 may include young men, etc.

Returning to FIG. 1, (e) Human-understandable clusters 118: After the profile-symptom-mitigation cluster creator 116 component creates the clusters, each cluster is related to a particular symptom with a series of mitigations and profiles associated with this cluster. These clusters are human understandable because humans can read all texts that belong to a cluster and inspect the patient centroids which generalize and describe every profile data input point that belongs to the cluster. It can be inspected by a human to understand what a typical patient in a particular symptom group looks like and which mitigation is rated as helpful by which type of patient.

(5) User Profile Compiler 120: The user profile compiler 120 component is used to create a profile for the user 122. In embodiments this can involve providing a form to the user 122 to provide the required information. Alternatively, a chatbot could be used to generate the profile. The user profile compiler 120 can be implemented by a module or component in embodiments.

(6) User Symptom Compiler 124: The user symptom compiler 124 component is used to describe the symptoms that the user 122 is experiencing. For example, this component could present a form to the user 122 that allows the user 122 to write free text into the form and choose a label from the label set defined in the creation of the clusters by the profile-symptom-mitigation cluster creator component 116.

(7) Profile and Symptom Matcher 126: The profile and symptom matcher 126 component transforms the information generated by the user profile compiler 120 component and the user symptom compiler 124 component to mirror the same input vectors provided by the profile-symptom-mitigation cluster creator 116 component. For example, user 122 may have provided input in a different format or order than the input vectors provided by the profile-symptom-mitigation cluster creator 116 component. The user 122 may provide gender first and then age whereas the input vectors provided by the profile-symptom-mitigation cluster creator 116 component may list age and then gender. To continue the example, the user 122 may provide F, to represent a gender of female, whereas the profile-symptom-mitigation cluster creator 116 component uses the text female. In embodiments a script may be used to correctly format and order the information generated by the user profile compiler 120 component and the user symptom compiler 124 component to mirror the same input vectors provided by the profile-symptom-mitigation cluster creator 116 component. The two distance measures defined by the profile-symptom-mitigation cluster creator component are used to assess first which level 1 clusters the user is closest to and second which level 2 cluster the user is closest to. The profile and symptom matcher 126 may be implemented by a component or module.

(f) Selected Cluster 128: This is the level 2 cluster selected by the profile and symptom matcher 126 component and is given as input to the output creator.

(8) Output Creator 130: For the user symptom and profile and the chosen level 2 cluster, the output creator 130 component computes the following output for the user 122:
a. Compute for each text entry in the cluster how close this entry is to the user symptom text entered by the user 122 as a first ranking. For this, it is possible to use a threshold and all entries too far away from the user symptom text based on the threshold are discarded. In an example, an LLM may encode each token/word in the text input from the user 122 and apply a pooling over the input to generate a fixed length vector that represents the meaning of the input. This can be performed for each input and using a defined similarity measure, such as a Euclidean distance, the entry with the lowest distance/highest similarity is used.
b. Compute for each profile in the cluster how similar the user profile is as a second ranking. For this, it is possible to use a threshold and all profiles too far away from the profile based on the threshold are discarded. Similarity and all profiles being too far away from the profile may utilize a similar LLM, fixed length vector, and similarity measure (e.g., Euclidean distance) described in a. above.
c. Combine the two rankings. How the combination is done depends on the use case or specific application. One example would be to add up the distances and divide by two (this would assume the same distance measure is used and they have the same meaning). It would also be possible to re-weight the distances (e.g., to give more weight to the first or second ranking).
d. Based on the combined ranking, the different mitigation strategies are compared and it is checked for each one if it is similar or the same to any of the others. For this, a threshold of top k entries could be used. In an embodiment, a database of synonyms can be used, for example to equate “using an ice pack” as being similar to “applying ice.” In embodiments, each mitigation strategy may be encoded with an LLM and then classified as being similar or not. A natural language inference model may be used to classify the mitigation strategies as being similar or the same to any of the others as well.
e. Based on the result of the comparison and check, the most common mitigation strategy that worked well is identified.

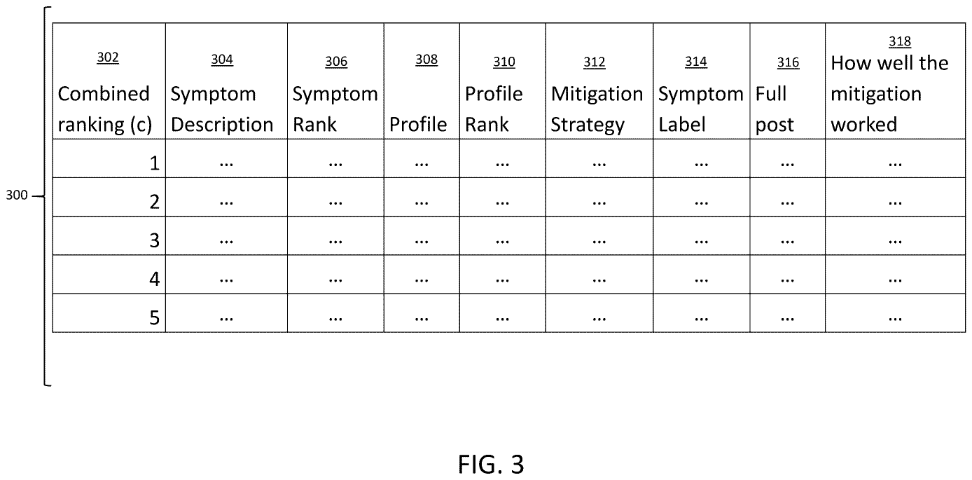

FIG. 3 illustrates how the above information from the output creator 130 component of FIG. 1 can be compiled and visualized, for example, displayed to the user on their electronic device.

(e) Explainable Disease Identification & Recommendation 132: The output to the user 122 can take the following form:
a. Returning the most promising mitigation strategy from the output creator 130 component (e.).
b. Outputting all posts which this mitigation strategy links to.
c. Indicating a profile match between the user and a poster profile for the chosen mitigation strategy output by the output creator 130 component (b.).

d. In some cases, the user can request more information and/or receive the table 300 shown in FIG. 3. For example, the table 300 can include a combined ranking 302, a symptom description 304, a symptom rank 306, profile 308, profile rank 310, mitigation strategy 312, symptom label 314, full post 316, and how well the mitigation worked 318. The profile 308 can include information from the profile DB 110 including information about the author of a particular post. The symptom label 314 may represent the label generated by the LLM from the clusters. The full post 316 and how well the mitigation worked 318 can be extracted from the post and any comments or replies to the post by the system 100. In embodiments, the combined ranking 302 may be an average rank of symptom description 304 and symptom rank 306. The symptom rank 306 may correspond to the corresponding rank of top k entries from the output creator 130. The profile rank 310 may correspond to a rank as described above such as computing for each profile in the cluster how similar the user profile is as the profile rank 310. For this, it is possible to use a threshold and all profiles too far away from the profile based on the threshold are discarded. The profile rank 310 may represent how similar two profiles are, for example, two profiles where both correspond to young women may be most similar with a corresponding higher profile rank 310.

Embodiments of the present invention thus provide for general improvements to computers in machine learning systems to predict a most promising mitigation strategy for a user's symptoms in an automated fashion using crowd sourced data that is accurate, reliable, trustworthy, explainable and transparent, overcoming technical obstacles such as the lack of heterogeneity in the text of posts and difficulties in computer understanding of the text, while at the same time providing personalized recommendations based on profile matching.

Moreover, embodiments of the present invention can be practically applied to use cases to effect further improvements in technical fields such as digital medicine and personalized healthcare.

In an exemplary embodiment, the AI method and system according to the present invention can be applied to provide automated and personalized advice for a patient. In this use case, a patient has a health issue with symptoms that he/she can describe. The patient interacts with the AI method and system according to the present invention to receive advice on how he/she can best mitigate the symptoms. As input, the AI method and system receives: (1) the patient enters in (a) information about his/her profile and (b) text describing his/her symptoms, and (2) social media/crowd source data. The AI method and system maps the patient's profile and symptoms to the recorded profiles and symptoms, identifying the closest match, describing why it is a match and offering the matching person's successful mitigation advice to the patient in a transparent manner. The output includes a text-based recommendation with evidence as an explanation for the recommendation.

In an exemplary embodiment, the AI method and system according to the present invention can be applied to provide a treatment recommendation for a doctor to a patient. In this use case, the patient has a health issue with symptoms and the doctor and/or patient interacts with the AI method and system according to the present invention to receive advice on how the patient can best mitigate the symptoms. The doctor might also be unsure how to best treat the symptoms. As input, the AI method and system receives: (1) the patient enters in (a) information about his/her profile and (b) text describing his/her symptoms, and (2) social media/crowd source data. The AI method and system maps the patient's profile and symptoms to the recorded profiles and symptoms, identifying the closest match, describing why it is a match and offering this matching person's successful mitigation advice to the doctor in a transparent manner. If the doctor does not see any issues with the advice, the doctor may pass it along to the patient. The output includes a text-based recommendation with evidence as an explanation for the recommendation.

The mitigation strategies which can be determined may be checked for safety, but are not limited and can include any treatment, exercise, food or diet plan, vitamin, supplement, medication (including prescription medications or drugs in some embodiments), etc. referenced by the social media/crowd sourced data.

In an embodiment, the present invention provides an AI method and system that searches social media/crowd sourced data to give advice to a user who inputs a particular set of symptoms by the following steps:
Setting up of a symptom DB with symptoms, aliases and associated diagnoses, for example from scientific publications (including reliability weighting) and databases (e.g., UMLS).
Obtaining all social media sources that are desired to use to be used to build the system, for example Reddit categories or hashtags about health, and storing the text in a social media DB.
Instantiating a post chooser component which decides whether a social media post from the social media DB is rejected or added to the system, in particular determining whether it contains desired information (symptom, mitigation) and passes safety filters.
Iterating over each social media post and:
   Building of a profile DB containing demographic information about the author by employing the social media profile extractor component. This information is stored in the profile DB.
   Building a symptom-mitigation database by extracting symptoms, relevant medical entities and mitigations from the post by the social media symptom extractor component. Symptoms are mapped to the symptom DB.
Creating a clustering of the profile-symptom-mitigation database by the profile-symptom-mitigation cluster creator component using the entries of the symptom-mitigation database. Inside of the symptom-mitigation-clusters, profile clusters are generated based on prototypes. This leads to human-understandable clusters. The profile clusters may be generated based on Prototype-based learning, where the prototype serves as a representative of the cluster. Similar to a KNN algorithm, the prototype may be the vector that represents the middle of the cluster. The profile clusters may be said to be “human-understandable clusters” as the system assigns new data points by closest distance (e.g., Euclidean distance) to a cluster. A given prototype of a cluster is a fictive profile that includes information such as gender: female, age: 25, etc. This profile is then “human understandable” as a prototypical representative of the cluster.
The user provides the system with the following information:
   Profile information (age, gender, diagnoses, etc.) for the user profile compiler component.
   Symptoms to be matched to the symptom DB by the user symptom compiler component.
Matching user profile and symptoms provided by the user to the closest cluster in the profile-symptom-mitigation database by the profile and symptom matcher component.
Computing of the output that will be provided to the user by the output creator component.
Generating the final output including an explainable disease identification and mitigation recommendation.

Embodiments of the present invention provide for the following improvements and technical advantages over existing technology:
1) Matching a user's symptoms via creating profile-symptom-mitigation clusters, which allows to improve accuracy based on the insight that people with different profiles might need different mitigation strategies for the same symptom.
2) Providing a two level clustering procedure that first clusters based on text, describing similar symptoms, together and then further divides each cluster, based on tabular data, depending on which profiles can be grouped together within the cluster.
3) Compiling specifically for a user and their matched level 2 cluster the information such that the most promising mitigation strategy is identified.
4) Generating a response with a specific structure for transparency, the output including the following information:
   a. Returning the most promising mitigation strategy from the output creator component (e.).
   b. Outputting all posts which this mitigation strategy links to.
   c. Indicating a profile match between the user and a poster profile for the chosen mitigation strategy output by the output creator component (b.).
   d. In some cases, the user can request more information and/or receive the table shown in FIG. 3.
5) Enabling to uncover the correct or most promising piece of information hidden in the vast amount of social media knowledge, which can improve a person's quality of life on how they can best mitigate their symptoms.

Existing methods for exploring the extraction of medical information and related user experiences shared on social media, among other technical deficiencies, are limited to well-defined medical problems like discovery of symptoms or medication effects that have not been observed before. In contrast, embodiments of the present invention introduce solutions to a problem that is very relevant for patients and aims to have an immediate impact on patient well-being compared to other approaches. Additionally, with patients as end-users, the system grants them direct control over mitigating their symptoms. At the same time, the AI method and system according to an embodiment of the present invention can also be easily adopted for doctors and provide information on a more detailed or terminology-specific level.

The AI method and system according to an embodiment of the present invention can be used in applications for the health literacy survey, or in applications directed to users to support their health and lifestyle (e.g., to prevent future diseases). The AI method and system according to an embodiment of the present invention could also be applied to support doctors with tailored information to help their patients, which is especially advantageous in case of rare diseases where the doctor is less experienced. Alternatively, health insurance companies could use it for prevention and risk mitigation. It could also be used to either obtain a second opinion or to avoid going to a doctor for an easily solved health issue.

In contrast to existing technology, embodiments of the present invention provide solution that can leverage a user's description of a profile and symptoms and then use social media posts to identify the disease and advice on how to mitigate symptoms. Further, in contrast to existing technology, embodiments of the present invention provide to compute:
A profile-symptom-mitigation matching, which enables to provide more accurate and personalized recommendations to users.
The two level clustering, which enables to identify which people match to which symptoms and similar people, therefore further improving the accuracy of the personalized recommendations.
The improved user personalized mitigation strategy along with a human-understandable explanation for it.

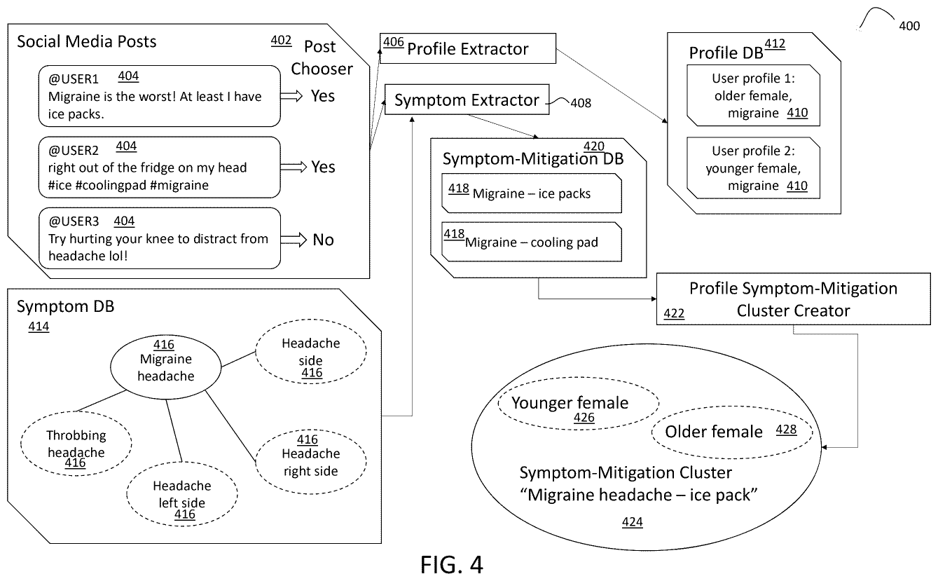

FIG. 4 includes workflow 400 for a method and system for processing social media posts and user profiles to create symptom-mitigation clusters according to an embodiment of the present invention. In embodiments, the post chooser 402 selects social media posts 404 which are provided to profile extractor 406 and symptom extractor 408. As described in FIG. 1, the post chooser 402 selects particular social media posts from a social media DB based on whether the posts include information about a possible way to mitigate a particular symptom. The profile extractor 406 may take a social media post 404 selected by the post chooser 402 and generates a profile 410 for the author of the social media post 404. The profiles 410 can be stored and accessed from profile DB 412.

The workflow 400 also includes symptom DB 414 which stores a list of possible symptoms 416 based on the UMLS. The dashed lines around symptoms 416 represents a more fine-grained example of a symptom. For example, “throbbing headache” may explain “migraine” in more detail. The symptom extractor 408 may be an example of the Social Media Symptom Extractor 112 of FIG. 1. The social media post 404 selected by the post chooser 404 may be transmitted to the symptom extractor 408 for extracting all symptoms and corresponding mitigation strategies 418 found in the social media post 404. As illustrated in FIG. 4, the symptoms and corresponding mitigation strategies 418 can be stored in the symptom-mitigation DB 420. In workflow 400 the profile symptom-mitigation cluster creator 422 can use the entries (418) of the symptom-mitigation DB 420 to generate symptom-mitigation clusters 424. For example, workflow 400 depicts symptom-mitigation cluster 424 for “migraine headache-ice pack.” Included in symptom-mitigation cluster 424 is various profile clusters 426 and 428.

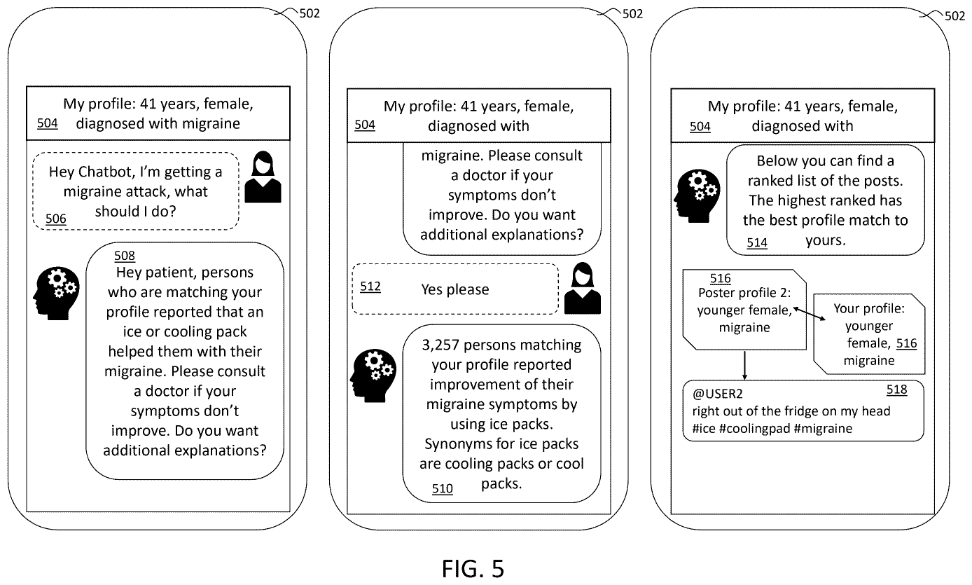

FIG. 5 illustrates an example output that could be presented to a user via a chatbot in a smartphone app according to an embodiment of the present invention. FIG. 5 depicts various outputs presented via user device 502. For example, the output can include information for the user, such as their profile 504. A user can provide input, such as user symptoms at 506. The current disclosure as described herein can use the information from the profile 504 and user symptoms 506 to generate identifications of diseases or conditions as well as recommendations for further treatment 508. The chatbot implemented by the current system described herein can provide the recommendations 510 based on further input 512 provided by the user. The output presented via user device 502 can also include a ranking 514 of user profiles 516, and posts 518 of those users, which match and/or are most similar to the user symptoms 506 and profile 504.

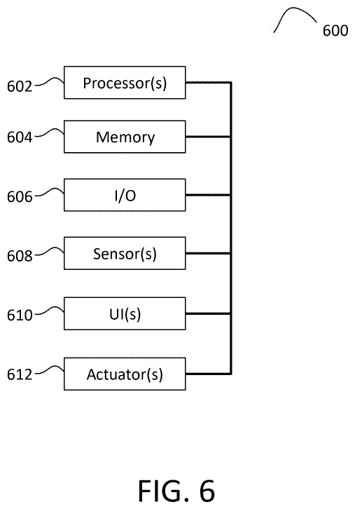

Referring to FIG. 6, a processing system 600 can include one or more processors 602, memory 604, one or more input/output devices 606, one or more sensors 608, one or more user interfaces 610, and one or more actuators 612. Processing system 600 can be representative of each computing system disclosed herein.

Processors 602 can include one or more distinct processors, each having one or more cores. Each of the distinct processors can have the same or different structure. Processors 602 can include one or more central processing units (CPUs), one or more graphics processing units (GPUs), circuitry (e.g., application specific integrated circuits (ASICs)), digital signal processors (DSPs), and the like. Processors 602 can be mounted to a common substrate or to multiple different substrates.

Processors 602 are configured to perform a certain function, method, or operation (e.g., are configured to provide for performance of a function, method, or operation) at least when one of the one or more of the distinct processors is capable of performing operations embodying the function, method, or operation. Processors 602 can perform operations embodying the function, method, or operation by, for example, executing code (e.g., interpreting scripts) stored on memory 604 and/or trafficking data through one or more ASICs. Processors 602, and thus processing system 600, can be configured to perform, automatically, any and all functions, methods, and operations disclosed herein. Therefore, processing system 600 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 600 can be configured to perform task “X”. Processing system 600 is configured to perform a function, method, or operation at least when processors 602 are configured to do the same.

Memory 604 can include volatile memory, non-volatile memory, and any other medium capable of storing data. Each of the volatile memory, non-volatile memory, and any other type of memory can include multiple different memory devices, located at multiple distinct locations and each having a different structure. Memory 604 can include remotely hosted (e.g., cloud) storage.

Examples of memory 604 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 604.

Input-output devices 606 can include any component for trafficking data such as ports, antennas (i.e., transceivers), printed conductive paths, and the like. Input-output devices 606 can enable wired communication via USB®, DisplayPort®, HDMI®, Ethernet, and the like. Input-output devices 606 can enable electronic, optical, magnetic, and holographic, communication with suitable memory 606. Input-output devices 606 can enable wireless communication via WiFi®, Bluetooth®, cellular (e.g., LTE®, CDMA®, GSM®, WiMax®, NFC®), GPS, and the like. Input-output devices 606 can include wired and/or wireless communication pathways.

Sensors 608 can capture physical measurements of environment and report the same to processors 602. User interface 610 can include displays, physical buttons, speakers, microphones, keyboards, and the like. Actuators 612 can enable processors 602 to control mechanical forces.

Processing system 600 can be distributed. For example, some components of processing system 600 can reside in a remote hosted network service (e.g., a cloud computing environment) while other components of processing system 600 can reside in a local computing system. Processing system 600 can have a modular design where certain modules include a plurality of the features/functions shown in FIG. 6. For example, I/O modules can include volatile memory and one or more processors. As another example, individual processor modules can include read-only-memory and/or local caches.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A computer-implemented method for determining optimized patient symptom-mitigation predictions, the method comprising:
receiving profile information and user symptoms from a user;
matching the profile information and the user symptoms to one of a plurality of profile clusters that are within one or more symptom-mitigation clusters created using textual data, the profile clusters being grouped within the one or more symptom-mitigation clusters based on profile information of authors of the textual data; and
predicting a symptom-mitigation strategy based on the matching profile cluster.

2. The computer-implemented method of claim 1, further comprising obtaining the textual data at least in part from social media posts and/or crowd-sourced data, and building a profile database including the profile information about the authors of the social media posts and/or the crowd-sourced data.

3. The computer-implemented method according to claim 1, further comprising:
building a symptom-mitigation database by extracting symptoms and mitigation strategies from the textual data, wherein the one or more symptom-mitigation clusters are generated using a clustering algorithm, the profile information from the profile database, and the symptom-mitigation database.

4. The computer-implemented method according to claim 2, wherein the textual data is taken at least in part from the social media posts, which are selected from a social media database by classifying the social media posts by patients from patient groups based on content of the social media posts.

5. The computer-implemented method according to claim 1, further comprising:
computing, for each text entry of the matching profile cluster, similarity to text of the user symptoms;
computing, for each profile in the matching profile cluster, similarity to the profile information from the user;
determining a combined ranking by combining the similarity to the text of the user symptoms and the similarity to the profile information from the user; and
identifying a most common symptom-mitigation strategy based on the combined ranking.

6. The computer-implemented method according to claim 5, further comprising outputting to the user the most common symptom-mitigation strategy along with the textual data that links to the most common symptom-mitigation strategy and profile matches.

7. The computer-implemented method according to claim 5, wherein the combined ranking is determined by averaging a distance determined for the similarity to the text of the user symptoms and a distance determined for the similarity to the profile information from the user, and wherein different mitigation strategies are checked to determine if they are similar in order to identify the most common symptom-mitigation strategy.

8. The computer-implemented method according to claim 1, further comprising filtering the textual data based on whether the textual data contains information about how to mitigate a symptom.

9. The computer-implemented method according to claim 1, further comprising filtering out, modifying or marking unsafe information in the textual data.

10. The computer-implemented method according to claim 1, wherein the clusters of profiles are generated based on prototypes.

11. The computer-implemented method according to claim 1, wherein the one or more symptom-mitigation clusters are created using disease or symptom labels, wherein textual descriptions of symptoms are encoded as vectors using a large language model (LLM), and wherein the encoded vectors are used to train a clustering algorithm.

12. The computer-implemented method according to claim 1, further comprising concatenating the profile information for each of the authors of the textual data into a feature vector, which in each case is assigned to a mitigation strategy extracted from the textual data, and training a clustering model for each of the one or more symptom-mitigation clusters to generate the plurality of profile clusters in the one or more symptom-mitigation clusters.

13. The computer-implemented method according to claim 1, further comprising generating a new feature vector based on a determination that a respective feature vector of the profile information of one of the authors has a distance to a centroid of one of the profile clusters that is greater than a threshold.

14. A computer system comprising one or more hardware processors which, alone or in combination, are configured to provide for execution of a method for determining patient symptom-mitigation predictions comprising the following steps:
receiving profile information and user symptoms from a user;
matching the profile information and the user symptoms to one of a plurality of profile clusters that are within one or more symptom-mitigation clusters created using textual data, the profile clusters being grouped within the one or more symptom-mitigation clusters based on profile information of authors of the textual data; and
predicting a symptom-mitigation strategy based on the matching profile cluster.

15. A tangible, non-transitory computer-readable medium having instructions thereon, which, upon being executed by one or more processors provide for execution of a method for determining patient symptom-mitigation predictions comprising the following steps:
receiving profile information and user symptoms from a user;
matching the profile information and the user symptoms to one of a plurality of profile clusters that are within one or more symptom-mitigation clusters created using textual data, the profile clusters being grouped within the one or more symptom-mitigation clusters based on profile information of authors of the textual data; and
predicting a symptom-mitigation strategy based on the matching profile cluster.

16. The computer-implemented method of claim 1, wherein the symptom-mitigation strategy is used to support decision making, and wherein the method is applied to optimize a healthcare machine learning or artificial intelligence system for a medical patient.
